(19) United States
(12) Patent Application Publication
(10) Pub. No.: US 2005/0096607 A1
(43) Pub. Date: May 5, 2005

(54) Hydrostream thrombectomy system

(76) Inventor: Robert C. Beck, St. Paul, MN (US)

Correspondence Address: Beck & Tysver, P.L.L.C., Suite 100, 2900 Thomas Avenue S., Minneapolis, MN, 55416, US

(21) Appl. No.: 10/922,003

(22) Filed: Aug. 19, 2004

Related U.S. Application Data
(63) Continuation-in-part of application No. 09/637,529, filed on Aug. 11, 2000.
(60) Provisional application No. 60/496,429, filed on Aug. 20, 2003.

(51) Int. Cl. A61M 5/00

(52) U.S. Cl. 604/264

(57) ABSTRACT

A catheter for interacting with occlusive material in a blood vessel having a Coanda nozzle driven by an injected fluid flow which mixes with occlusive material to ablate and macerate the occlusive material.

### CROSS REFERENCE TO RELATED CASES

The present case is a continuation in part of U.S. Ser. No. 09/637,529 filed Aug. 11, 2000 which is incorporated by reference in its entirely. The present case is the utility case based upon Provisional Application U.S. Ser. No. 60/496,429 filed Aug. 20, 2003 which is incorporated by reference in its entirely.

### FIELD OF THE INVENTION

The present invention relates generally to catheter based therapeutic system and more particularly to a thrombectomy catheter system.

### BACKGROUND OF THE INVENTION

Thrombectomy catheters are known in the art from U.S. Pat. Nos. 5,320,599; 5,370,609 and 5,344,395 among others. Recently available products include the “Oasis” from Boston Scientific, the “Hydrolyser” from Cordis, and the “Angiojet” available from Possis. Each of these devices uses the energy from an injected stream of fluid to aspirate and interact with clot or occlusive material and to remove it from the body. In this respect, each of these prior art devices have at least two lumens, including a fluid supply lumen and a discharge or exhaust lumen. Each of these devices also includes one or more retrograde directed jets that form an ejector configuration in the distal tip of the device. In some instances (Hydrolyser and Angiojet) the jet is shrouded from the clot to protect the vessel. In the case of the Oasis catheter, the jet is directly exposed to the clot. These structural differences result in clinical performance differences.

### SUMMARY OF THE INVENTION

The present invention includes a Coanda nozzle located in the distal tip of the catheter. Fluid under pressure is supplied to the Coanda nozzle from an angiographic injector or other pump power source. In some embodiments, the Coanda nozzle jet is directly exposed to the dot or occlusive material. In an alternative embodiment the Coanda nozzle jet is shrouded. In this shrouded version the jet induces and directs a secondary or combined flow into the catheter discharge sheath that forms the shroud. The discharge sheath of the device may be fixed with respect to the Coanda nozzle or the Coanda nozzle and the discharge sheath may be movable with respect to each other.

### BRIEF DESCRIPTION OF THE DRAWINGS

Through out the several figures identical reference numerals indicate equivalent structures, wherein:

FIG. 1 is a diagram of the distal tip of a representative embodiment of the fluid supply catheter;

FIG. 2 is a drawing of a movable open tip sheath embodiment of the system;

FIG. 3 is a drawing of a movable open tip sheath embodiment of the system;

FIG. 4 is a drawing of a recirculation sheath embodiment of the system;

FIG. 5 is a drawing of a recirculation sheath embodiment of the system;

FIG. 6 is a drawing of a recirculation sheath embodiment of the system;

FIG. 7 is a drawing of an embodiment of the system;

FIG. 8 is a drawing of an embodiment of the system;

FIG. 9 is a drawing of an alternate embodiment of the device;

FIG. 10 is a drawing of an alternate embodiment of the device;

FIG. 11 is a drawing of an alternate embodiment of the device;

FIG. 12 is a drawing of an alternate embodiment of the device; and,

FIG. 13 is a drawing of angular relationships of an embodiment of the device.

### DETAILED DESCRIPTION

Coanda Effect

A complete understanding of the device and its operation is facilitated by a brief discussion of the Coanda effect which is named for a Romanian aviation pioneer. In 1910 Henri Coanda built an airplane powered by a piston engine that had fuel injected into an exhaust system where the fuel was burned to create “thrust”. He mounted deflector plates at the exhaust outlets to direct exhaust flow away from the fuselage of the plane and he was surprised when the plates caused the exhaust to be attracted to the fuselage. He spent much of his career studying the physics and fluid dynamics that resulted from this observation. The effect has been named after him and in his honor.

The Coanda effect can take place when an energetic jet of fluid is injected into a more static reservoir of fluid. In general, a jet of fluid entering a quiescent fluid entrains fluid and mixes with the quiescent fluid through a momentum exchange process. The high velocity input stream “widens” and slows down as the jet moves into the ambient fluid and interacts with the stationary fluid surrounding it. In a Coanda nozzle the amount of fluid that the jet can entrain is typically limited by a physical barrier that forms a wall near the input high energy jet or fluid stream. This wall on one side of the input stream forms an asymmetric nozzle configuration. The entrainment process evacuates this wall region and a localized low pressure zone is created. Some investigators call this low pressure area a “separation bubble”. The ambient pressure on the “other” or non-wall side of the jet is higher and it “pushes” the developing jet toward the separation bubble against the wall and the jet flows along the wall. The degree of turning can be controlled and degrees of turn can be large with 180 degrees being easily achieved. In the embodiments shown the turning angle is about 90 degrees but other larger and smaller turning angles are contemplated within the scope of the various embodiments. Wall geometry may be varied as well. Both conical and hemispheric wall surfaces are shown but more complex parabolic surfaces or multiple step faceted surfaces may be used as well.

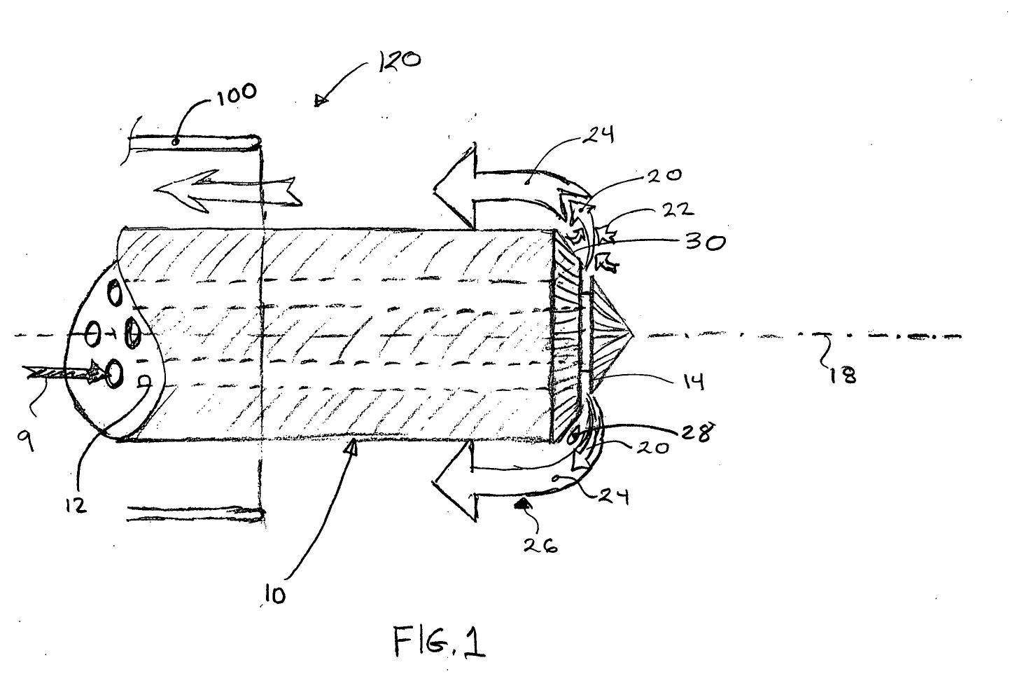

With respect to FIG. 1 the input fluid stream 9 supplied to the fluid supply lumens of the Coanda effect catheter 10 enters the side lumens typified by side lumen 12. The flow in the several fluid supply lumens merge at the slit 14 cut into the conical distal tip of the catheter. The primary fluid stream that emerges from the tip 20 is essentially a radial disk in the plane perpendicular to axis 18. If operated in air the fluid emerging 20 from the slit 14 forms a disk but when submerged in fluid, the primary jet 20 entrains ambient fluid indicated by arrows like arrow 22. The entrainment process is a momentum exchange process that increases the width of the combined flow indicated by arrow 24. The same process is occurring all around the nozzle but it is depicted somewhat differently in connection with the flow arrow 26. In this depiction the core stream 20 is shown “buried” within the combined flow 24. In the drawing the primary stream 20 picks up ambient fluid 22 and forms a combined flow 24 that follows the contour of a wall 30 as seen by the direction of flow arrow 26.

The entrainment process gives rise to the low pressure zone 28 or separation bubble near the wall 30 of the nozzle. It is the higher ambient pressure that forces the combined flow 26 to follow the contour of the wall surface 30. Although a conical tip with a straight wall 30 is depicted in most of the figures for simplicity, hemispheric or other more complex shapes are possible and desirable for some applications. The angle between the plane of the jet and the wall is about 45 degrees in the figure but other angles are desirable and operable as well. When the jet attaches to the wall it turns through about a 90 degree angle.

Structure of Fluid Delivery Catheter

FIG. 1 shows a composite interventional device 120 that comprises a inner fluid delivery Coanda effect nozzle catheter 10 and an overlying discharge sheath 100. In some embodiments the sheath 100 is optional or its function carried out by another structure like a procedure guide sheath or sub selective guide sheath. In some embodiments the sheath is fixed and in other embodiments the sheath is moveable with respect to the Coanda effect nozzle catheter 10.

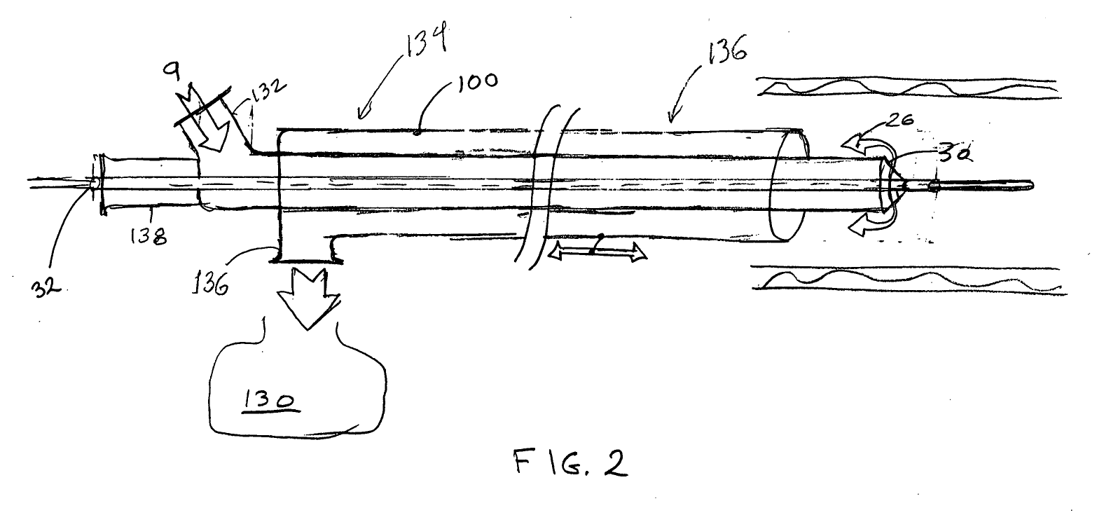

In this FIG. 1 example, an inner fluid delivery nozzle catheter 10 and the discharge sheath 100 are substantially concentric with each other and with the major axis 18 of the fluid delivery nozzle catheter. As seen in the figure a source of fluid under pressure 9 is provided and fluid is injected into the delivery lumens typified by lumen 12. Saline lytic drugs as well as CO2 laden fluids and contrast agent are acceptable fluids. A center lumen 32 seen in FIG. 2 may be provided to accept a guide wire. The slit 14 should be narrow and it is used to meter flow in the device. In general a slit with of 0.0005 to 0.005 inches is sufficient and a flow rate of between about 0.5 ml/sec to about 5 ml/sec are acceptable parameters. Although a slit is seen in this figure slots and holes are also very effective and useful as described later.

In general, a Coanda nozzle is provided near the distal tip of the catheter device and it induces a secondary flow in the blood vessel. In most embodiments the secondary flow is largely retrograde. This secondary flow may be recovered in a discharge sheath 100 and guided out from the body. The device may work alone without an exhaust sheath where the fluid delivery catheter 10 serves to emulsify dot or occlusive material. In this instance the saline or other suitable fluid is used to power the device. When used alone the saline fluid may be loaded with a thrombolytic drug to augment the treatment and render the debris more benign.

Structure of the Discharge Sheath Embodiments

In many embodiments the discharge sheath 100 forms a part of the overall system. In some instances the guiding catheter used to guide the fluid delivery catheter 10 to the site of intervention can also function as the discharge sheath.

FIG. 2 is representative of a preferred embodiment and it shows the discharge lumen of the discharge sheath 100 terminated at its proximal end in a collection bag 130. In operation, fluid 9 is delivered to the injection lumen 132 located on the proximal end 134 of the system. This fluid flows down the catheter to the Coanda nozzle at the distal tip where it initially emerges in an approximately radial direction. This is referred to through out as the “emerging jet”. The wall surface 30 encourages attachment and as the emerging jet develops as show by arrow 26 the jet is deflected through about ninety degrees. The fully developed jet next flows into the discharge sheath 100. After the flow has moved about 10 nozzle slot or slit widths from the exit plane of the emerging jet it is much slower and bigger and this condition is called the “fully developed” jet. Flow depiction arrow 26 is intended to show the path of the fully developed flow in the figure.

A high pressure fluid injector (not seen) is attached to connection 132. In many embodiments a guide wire lumen 32 is provided in the device and a guide wire coupler 138 is provided on the proximal section 134. The collection bag 130 is coupled to a coupler 136.

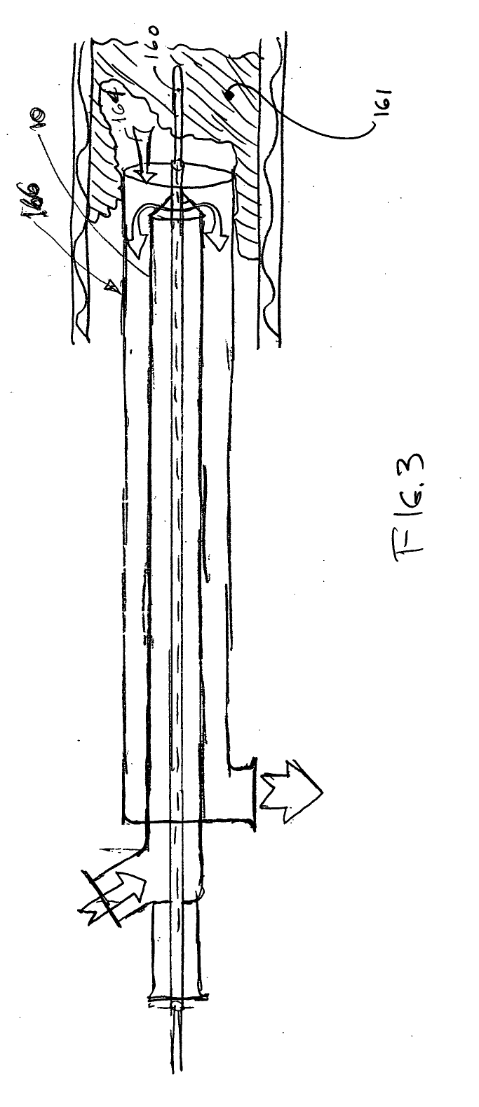

In FIG. 3 the sheath 100 has been advanced over the catheter nozzle section. In use the embodiment of FIG. 3 the guidewire 160 the sheath 100 and the fluid delivery catheter 10 are all capable of independent motion with respect to each other. Ambient fluid 164 can enter the open end of the distal section 166. In use the physician can advance the sheath to the location of the dot over the guide wire 160. Next the device 10 can be advanced to the dot 161 and turned “on” with the injection of fluid. As an alternative, the device 10 may be pushed through the dot 161 and then turned “on” and drawn back through the dot 161 into the sheath 100.

In use the physician may advance the sheath to an occlusion like a clot in the vessel by sequentially advancing the sheath and then the catheter 10. Both the sheath 100 and device 10 may be advanced and plunged into the dot. The fluid may be “on” or the device may be activated after it is inserted into dot. The sheath may be repeated advanced to cover over the fluid discharge nozzle or Coanda section and then retracted while the device itself remains stationary in the vessel.

The two figures seen as FIG. 3 and FIG. 2 taken together show one configuration and method of using the system. These figures show how the movable sheath embodiment of the system with both a Coanda effect catheter device and open sheath can work together to remove clot or other occlusive material.

Structure of Exhaust Sheath Section of the Catheter System

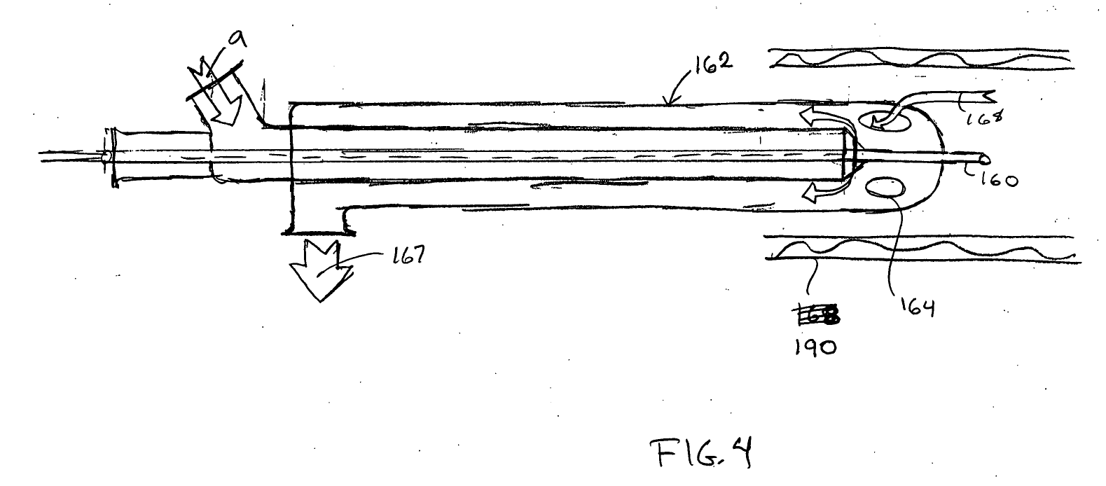

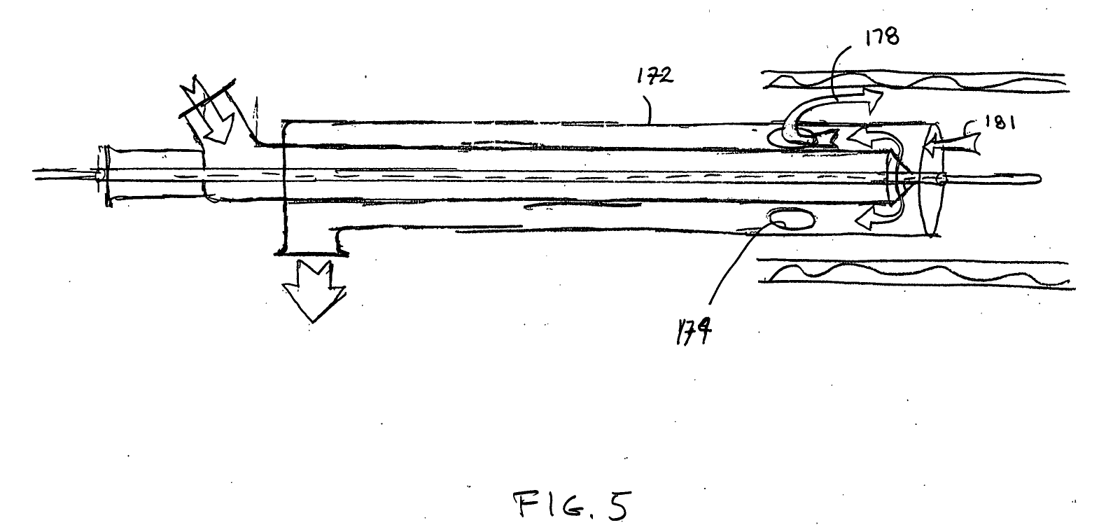

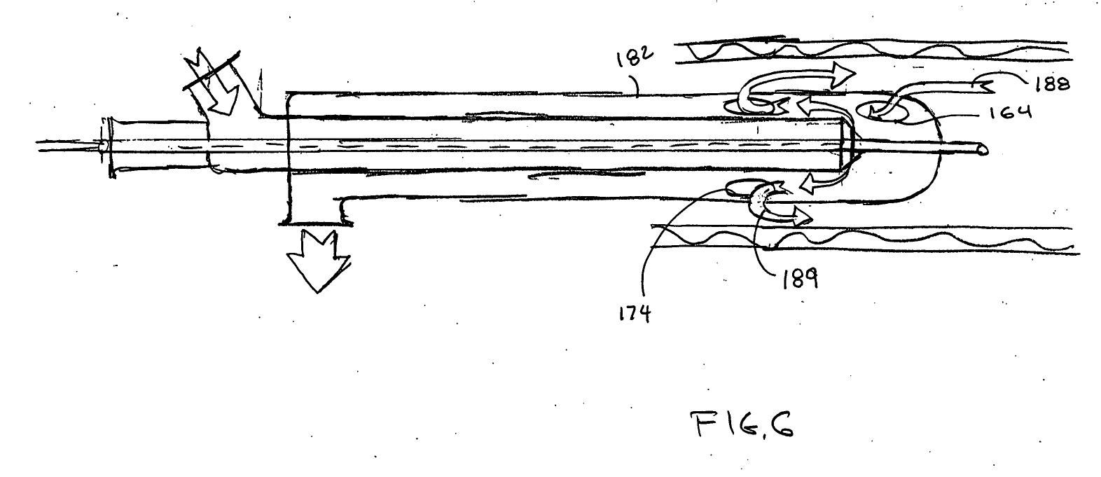

When the inner Coanda effect fluid delivery catheter 10 is operated inside of a sheath 166 (FIG. 3), the devices together form an effective device to emulsify and evacuate clot. The sheath may take any of several configurations. Several configurations are set forth in FIG. 4, FIG. 5 and FIG. 6. In these figures the proximal end of the catheter system is the same. For example for example each distal tip configuration and it is shown with a guidewire lumen connection 138 and fluid supply lumen connection 132 and an exhaust lumen connection 136. The figures show differing configurations of the distal tip.

In general the simplest and a preferred configuration for sheath 100 is an open ended tube which may be fixed to or attached to the inner fluid delivery catheter 10 or it may be moveable with respect to the inner catheter and the discharge sheath may operate in three separate situations as seen in FIG. 2 and FIG. 3.

FIG. 4 shows a closed end sheath 162 the sheath may have one or more open ports typified by port 164. These apertures may be on the rounded closed tip or set back doser to the Coanda nozzle. In this configuration the apertures are all distal of the slit in the Coanda nozzle. This position exposes the aperture to the low pressure zone created by the Coanda nozzle. Clot and other occlusive material is drawn in to the apertures from the vessel 190 as indicated by flow arrow 168. All of this occlusive material exits from the system through exhaust flow 167.

FIG. 5 shows an open-ended sheath 172 the sheath may have one or more open ports typified by port 174. In this configuration the apertures are all proximal of the slit in the Coanda nozzle. This position exposes the aperture to the higher pressure zone created by the Coanda nozzle. Clot and other occlusive material is drawn in to the open tip and partially discharged through the apertures as indicated by flow arrow 178. The material leaving the sheath through the proximal aperture s 174 is re-circulated further emulsifying the dot by flowing antegrade and reentering the device at the open tip depicted as arrow 181.

FIG. 6 shows a dosed end sheath 182 the sheath may have one or more open ports. These apertures may be on the rounded closed tip or set back closer to the Coanda nozzle. In this configuration the one set of aperture typified by port 164 is distal of the slit in the Coanda nozzle. Another set of apertures typified by port 174 is proximal of the slit in the Coanda nozzle. Apertures both in front of the Coanda nozzle and behind the Coanda nozzle causes clot and other occlusive material to be drawn in to the apertures as indicated by flow arrow 188. A portion of the material exits the sheath through proximal apertures such as 174. This combination of proximal and distal apertures creates a recirculation flow indicated by flow arrow 189.

Thus in the case of the closed tip the side apertures may be distal or ahead of the Coanda section or proximal of the Coanda section. With both proximal and distal multiple ports recirculation of secondary flow can occur.

Dynamic Properties of the Fluid Delivery Catheter

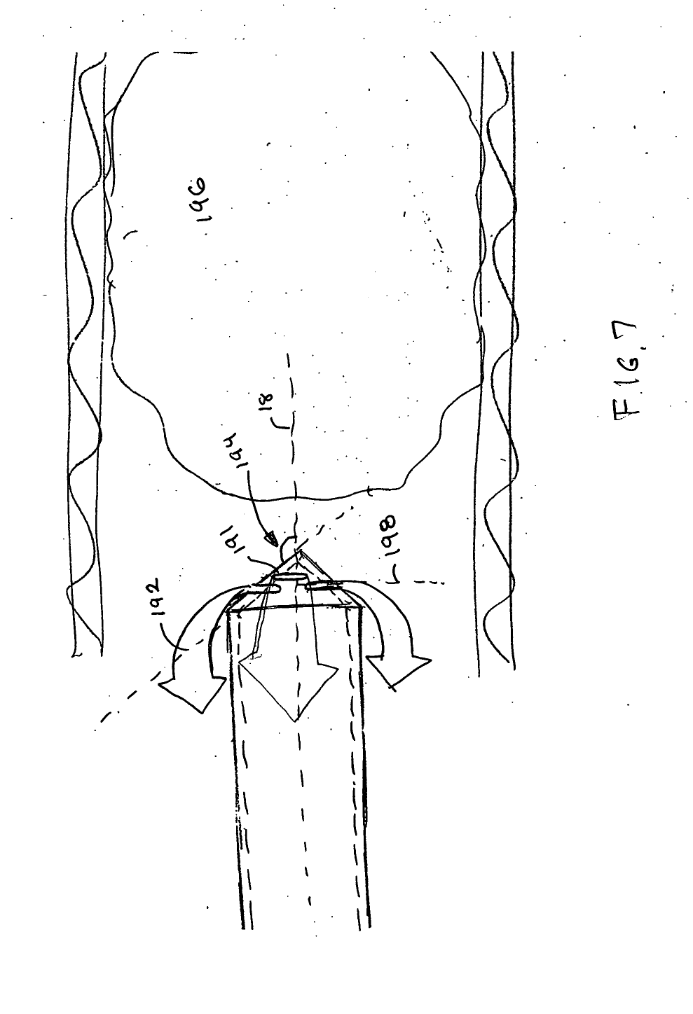

FIG. 7 and FIG. 8 should be considered together. FIG. 7 shows a fluid delivery catheter similar to that of FIG. 1 with the exception that the slit is replaced with a series of slots or holes typified by hole 191. Multiple slits may form two or more rings of slots around the circumference of the distal tip of the catheter. The catheter 10 uses an asymmetric nozzle and the Coanda effect at its distal tip to turn fluid through an angle so the injected flow is diverted from an approximately radial direction to a substantially retrograde direction depicted by flow arrow 192. The angle of the wall surface 194 with respect to the center axis 18 and the injection jet direction 198 work together to provide a switching action when the device is pressed into to dot 196, as further described in connection with FIG. 8. In general the angle between the jet as it emerges (198) and the wall surface (194) can vary from about zero degrees where the jet is tangent to the wall to about forty-five degrees. This wall angle may be constant or variable and it has an impact on the conditions causing the emerging jet to detach from the wall. The flow arrow 192 among others describe the steady state conditions that obtain in the unobstructed vessel. A different set of operating conditions apply when the device approaches and or enters a clot. In FIG. 8 the Coanda flow 26 is “switched off” the wall 194. The figure is intended to show conditions that result in the jet coming off the “wall”. In this situation the jet is very unstable and it will typically be in either the dot as seen in FIG. 8 or “on the wall” seen in FIG. 7. The FIG. 8 conditions should be understood to be transitory.

In this condition the catheter is in a dynamic state and the catheter can be considered “smart” in the sense that the device injects fluid into the dot when the jet is off the wall and is directly emulsifying clot. FIG. 8 shows the distal tip of the inner catheter 10 plunged into a dot where the injected fluid emerges and cuts a channel in the clot depicted by the dotted zone 199. It is important to note that the clot is dissected by the jet and that the highest energy in the jet is closet to the centerline or axis 18 of the device.

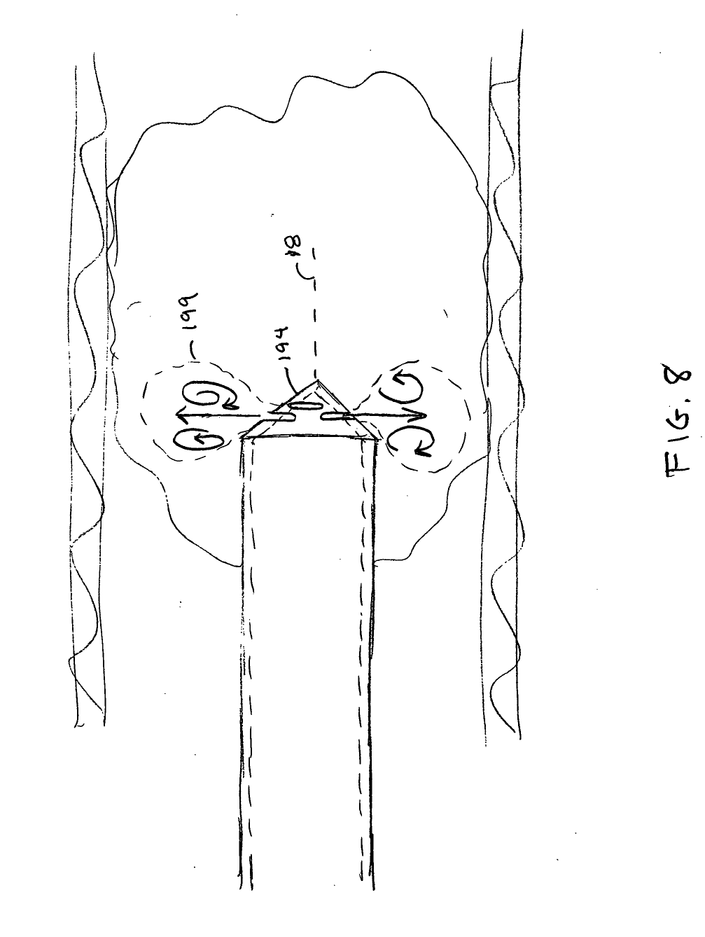

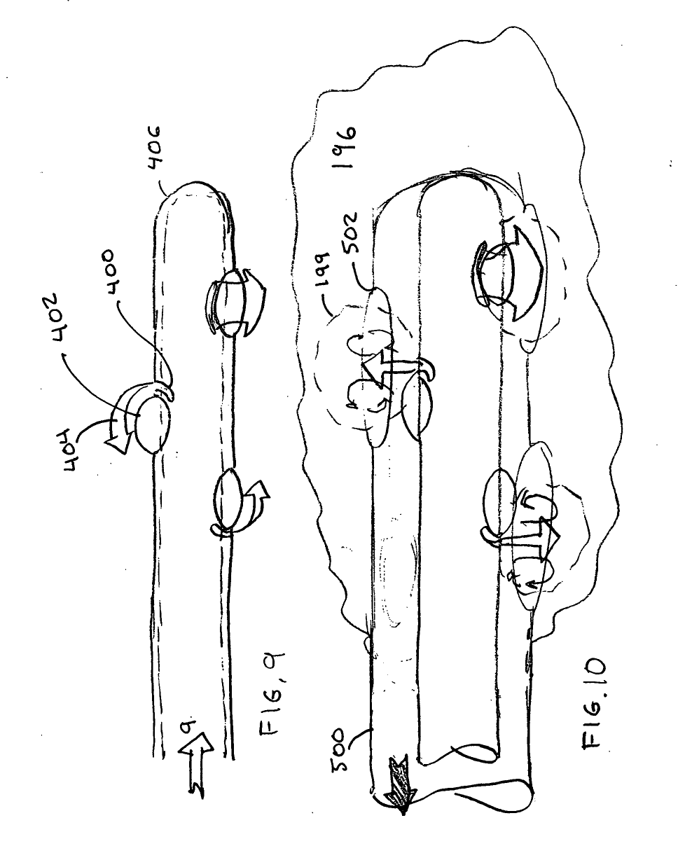

FIG. 9 and FIG. 10 should be considered together. In these embodiments several wall surfaces are positioned along the length of a catheter body. In this embodiment the catheter body 406 maybe a hypo tube of Nitinol or other alloy. Small raised bumps 402 are formed on the catheter body. These may be a separate injection molded plastic part or they may be formed in the wall of the tubing. These bumps are generally hemispheric is shape and at the base of each bump is a slit 400. The slit or slot communicates with the interior fluid 9 delivery lumen of the device. Fluid emerging from these slits or slots attaches to the bumps because of the Coanda effect. These bumps may be staggered along the length of the device and the slit need not align in one direction. For example they may direct fluid in a spiral path around the device or they may direct flow antegrade or retrograde or any combination of directions.

It is expected that this embodiment would be used for clearing dot from stroke victims or acute myocardial infarction patients. It is anticipated that the injected fluid would be physiologic saline alone or carrying a dose of a thrombolytic agent or perhaps CO2 as a contrast agent. In the cerebral arteries and small blood vessels it is hard to navigate with a sheath structure so this embodiment may be used without any effort directed at recovering the debris emulsified by the jets. Or as an alternative an integral or moveable sheath with holes 502 as seen in FIG. 10 may encase the device of FIG. 9 and form a discharge sheath 500 to remove clot 196 material from zones 199 formed in the clot.

As described in more detail in connection with FIG. 8 when the Coanda sections are plunged into dot the jets will detach from the hemispheric bumps and aggressively interact with the clot which is also depicted in FIG. 10.

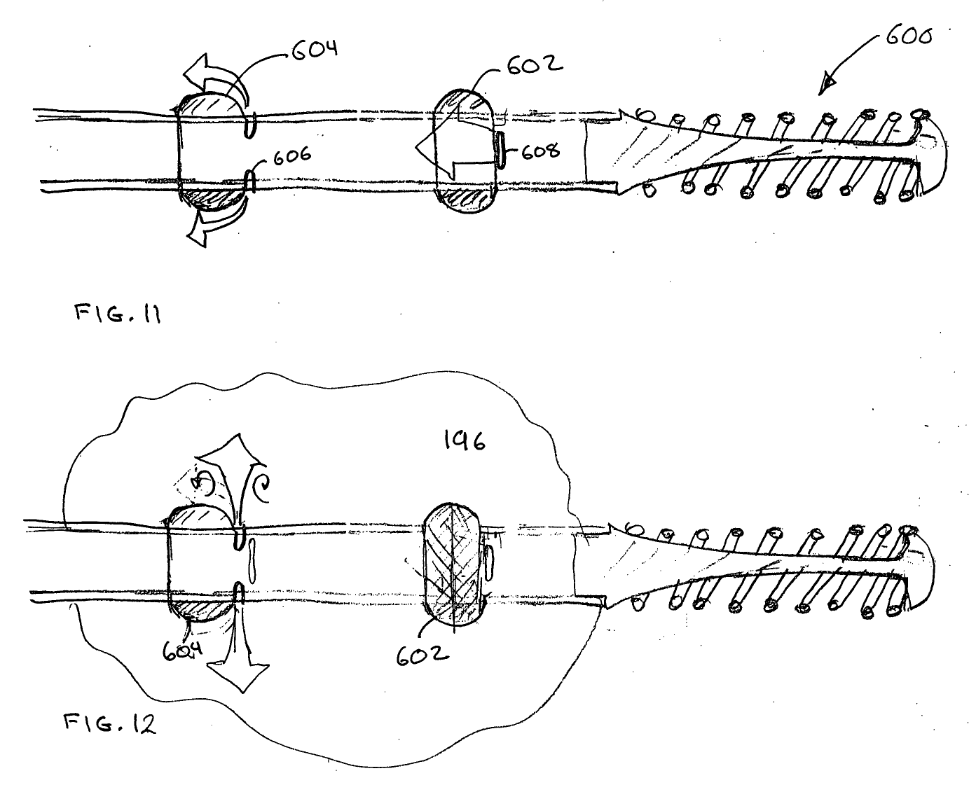

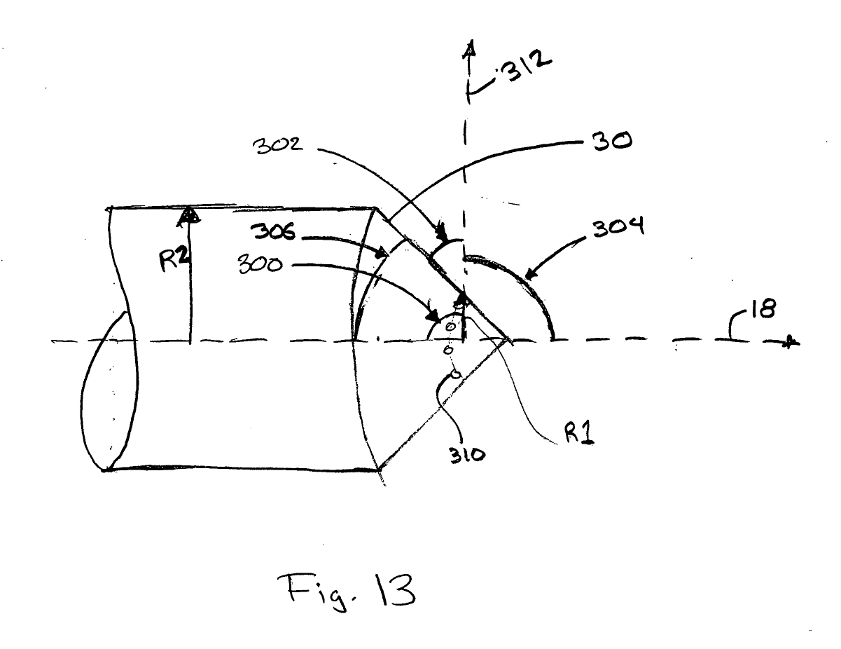

FIG. 11 shows an alternate embodiment of the device that may be used as a guidewire and or used to treat ischemic stroke. The distal tip has a conventional guide wire tip 600 formed at the end of an elongate hypo tube. Nitinol, stainless steel or other materials may be used for the elongate tubular body. In this embodiment several nubbins 604 and 602 are placed proximate slits 606 and 608 that together form Coanda wall attachment nozzles. When in clot 196 (FIG. 12) the nozzles are directed radially and interact aggressively with dot as seen in FIG. 12 and while the Coanda nozzles are in blood (FIG. 11) they lay down and follow the contour of the nubbin. The deflected jet should spread its energy over the surface of the nubbin and not be a hazard to the endothelial layers of the vessel.

FIG. 13 is intended to make clear the geometry relationships that gives rise to the Coanda effect. In the figure a series holes typified by hole 310 are arrayed around the body of the catheter. These holes intersect with the wall 30 of the tip at a radius R1. The jets which emerge from the holes are directed in a direction depicted by line 312. Angle 300 is the primary jet angle and in the figure it is about 90 degrees. The jet angle may vary from nearly 0 degrees to well over 90 degrees. The wall 30 is inclined with respect to axis 18 at an angle depicted by angle 306. The wall inclination angle in the figure is about 45 degrees and the wall angle may have a wide range of values. The angle between the wall and the jet or wall/jet angle is depicted as angle 302 and it may be zero up to about 60 degrees The angle in the figure is 45 degrees and this value works well. The jet emerges from the holes and attaches to wall 30 turning in the example through an approximately 90 degrees angle. As the primary jet mixes with blood and occlusive material it moves outwardly following the wall to the radius R2. Since the illustrated wall is a cone shape the area of the wall increases along the direction that the mixed flow follows. This feature means that the energy in the jet per unit area falls off as the jet moves from the radius R1 to R2. The geometry of the wall allows the designer to force the jet velocity or energy level to decay very fast which makes the device effective and efficient in the blood vessels. A high energy jet at radius R1 becomes a low energy jet at R2 as the jet is forced to spread out on the wall 30.

1. A Coanda effect catheter comprising:
a catheter body having at least one aperture directing fluid in a first jet direction;
a wall proximate the aperture at a wall/jet angle larger than 0 degrees and less than 60 degrees.

2. The Coanda effect catheter of claim 1 wherein:
said wall is a surface of revolution about an axis aligned with the catheter body;
whereby the area of the wall surface increase in the direction of flow along the wall.

3. The Coanda effect catheter of claim 2 wherein said wall surface is approximately conical.

4. The Coanda effect catheter of claim 2 wherein said wall surface is approximately spherical.
